(19) United States
(12) Patent Application Publication
(10) Pub. No.: US 2026/0207180 A1
(43) Pub. Date: Jul. 23, 2026

(54) SILICONE SPERM EXTRACTOR

(71) Applicant: Hong Li, Shaoyang (CN)

(72) Inventor: Hong Li, Shaoyang (CN)

(21) Appl. No.: 19/033,418

(22) Filed: Jan. 21, 2025

(51) Int. Cl. A61B 10/00; A61H 19/00

(52) U.S. Cl. CPC A61B 10/0058; A61H 19/32; A61H 2205/087

(57) ABSTRACT

A silicone sperm extractor is provided, which includes a silicone torso. The silicone torso is divided into lower limbs and a waist, a position between the lower limbs is filled with an automatic mechanism. The automatic mechanism includes an upper shell and a lower shell; the upper shell and the lower shell are detachably connected by bolts. Outer parts of the upper shell and the lower shell both abut against inner walls of the lower limbs of the silicone torso. An interior of an inner wall of the lower shell is fixedly connected to a power source, one side of the inner wall of the lower shell close to the power source is fixedly connected to a motor, a cavity container is driven to reciprocate by a plurality of connection rods outside the motor, allowing a user to have a comfortable experience during an extract semen operation.

### TECHNICAL FIELD

The present disclosure relates to the field of daily necessities technologies, and in particular, to a silicone sperm extractor.

### BACKGROUND

Silicone sperm collector is a device used to assist men in semen collection, typically used in medical, research, or fertility assistance settings. Its design aims to provide a comfortable and safe way to collect semen samples when needed. Silicone sperm collectors typically use high-quality silicone materials due to their good softness, elasticity, and durability, which can simulate the touch of human skin and provide a more comfortable experience. The device is typically designed to be ergonomic, adaptable to the physiological structure of men, and provide appropriate support and stimulation.

In the existing technology, some sperm collectors are fixed on a top of the base and a channel imitating female organs is set inside the silicone gel, grooves and protrusions are set inside the channel to increase stimulation, and then a user applies force to simulate sexual activity, thereby achieving a goal of sperm retrieval.

However, in this process, this single method and the need for self-exercise can easily cause the user to lose sexual desire, prolong the time for sperm retrieval, and even lead to organ weakness, rendering it difficult to complete sperm retrieval smoothly.

### SUMMARY

The present disclosure proposes a silicone sperm extractor, which aims to improve a problem of some devices in the prior art not being smooth enough during a sperm extraction process and easily causing a user to become weak.

To achieve the above objectives, the present disclosure provides the following technical solutions.

A silicone sperm extractor, including a silicone torso, where the silicone torso is divided into lower limbs and a waist; a position between the lower limbs is filled with an automatic mechanism, and the automatic mechanism includes an upper shell and a lower shell; the upper shell and the lower shell are detachably connected by a plurality of bolts; outer parts of the upper shell and the lower shell both abut against an inner wall of the lower limbs of the silicone torso; an interior of an inner wall of the lower shell is fixedly connected to a power supply; one side of the inner wall of the lower shell close to the power supply is fixedly connected to a motor; the motor is provided with a motor protection sleeve, and the protection sleeve is provided with through holes on top and bottom; the power supply and the motor are connected by wires; an output end of the motor is fixedly connected to a cam, an inner side of the cam is connected to a noise reduction gear; an outer side of the cam is rotatably connected to a connection rod, an interior of the connection rod is connected to two transmission wheels, one of transmission wheels is fixedly connected to one side of the cam, and an interior of the other transmission wheel is rotatably connected a cavity container; an outer part of the cavity container is slidably connected to on an inner wall of the lower shell; the cavity container is fixedly connected to two transmission rods, an outer side of the transmission rods is slidably connected at an interior of the through holes; an interior of the cavity container is filled with a silicone simulation organ, and an inner wall of the silicone simulation organ is provided with a plurality of protrusions.

In some embodiments of the present disclosure: a controller is fixedly connected to one side of the waist of the silicone torso; a signal line is fixedly connected to an output end of the controller; the other end of the signal line is fixedly connected to the motor inside the automatic mechanism, and the signal line is filled inside the silicone torso.

The present disclosure has the following beneficial effects:
in the present disclosure, by simulating human skin, a strong tactile sensation is formed for the user, and a silicone simulation organ is filled inside. An automatic mechanism is provided outside the silicone simulation organ, a cavity container is driven to reciprocate by a plurality of connection rods outside the motor. A plurality of protrusions are provided on an inner wall of the silicone simulation container, allowing the user to have a comfortable experience and smoothly and safely extract semen during operation.

### BRIEF DESCRIPTION OF DRAWINGS

FIG. 1 is a perspective view of a silicone sperm extractor proposed by the present disclosure.

FIG. 2 is a cross-sectional structure view of a silicone torso of the silicone sperm extractor proposed by the present disclosure.

FIG. 3 is a schematic structural diagram of a cavity container of the silicone sperm extractor proposed by the present disclosure.

FIG. 4 is a schematic structural diagram of a cam of the silicone sperm extractor proposed by the present disclosure.

FIG. 5 is a schematic structural diagram of a silicone simulation organ of the silicone sperm extractor proposed by the present disclosure.

FIG. 6 is a schematic structural diagram of a noise reduction gear of the silicone sperm extractor proposed by the present disclosure.

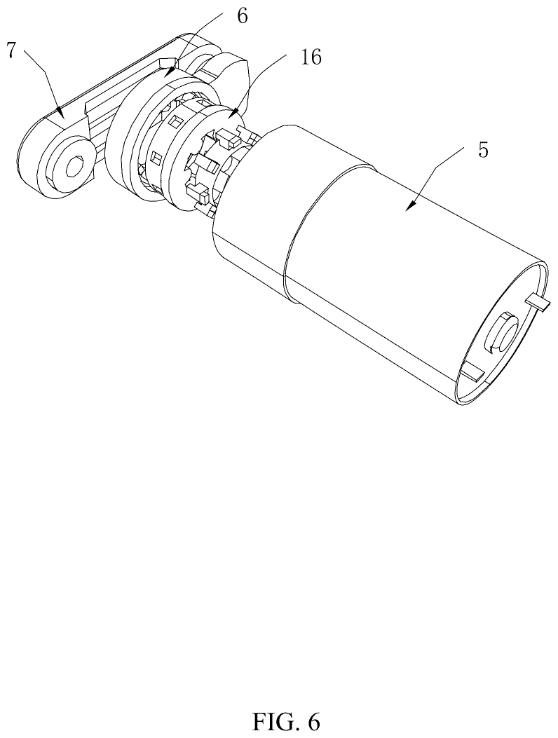

Numeral reference: 1—silicone torso; 2—upper shell; 3—lower shell; 4—power supply; 5—motor; 6—cam; 7—connection rod; 8—transmission wheel l; 9—cavity container; 10—transmission rod; 11—through hole; 12—controller; 13—signal line; 14—silicone simulation organ; 15—protrusion; 16—noise reduction gear.

### DESCRIPTION OF EMBODIMENTS

The following will provide a clear and complete description of the technical solution in the embodiments of the present disclosure, based on the accompanying drawings. Obviously, the described embodiments are only a part of the embodiments of the present disclosure, not all of them. Based on the embodiments in the present disclosure, all other embodiments obtained by those skilled in the art without creative labor are within the protection scope of the present disclosure.

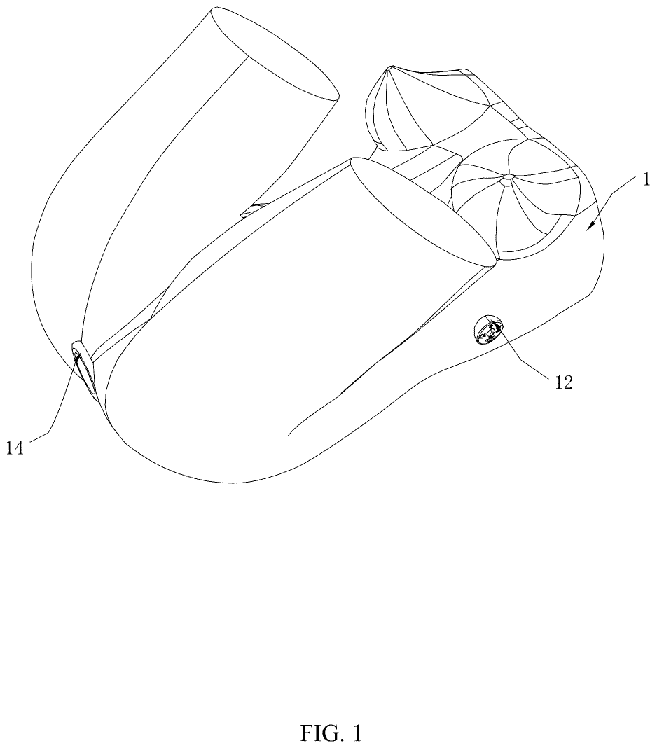

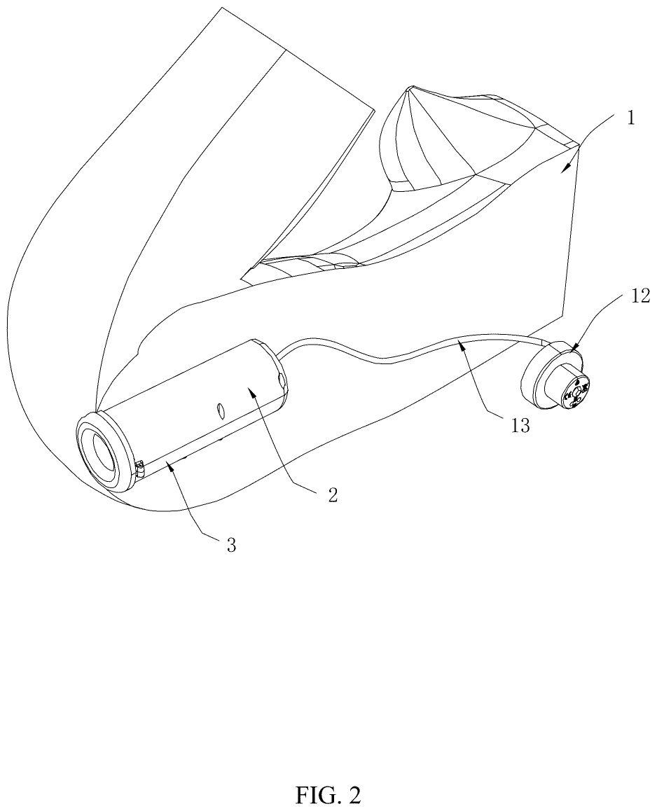

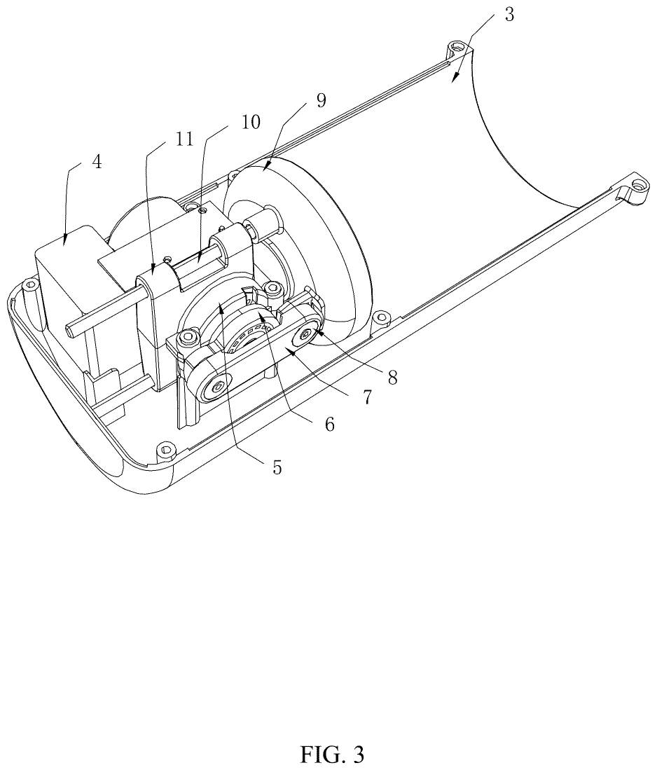

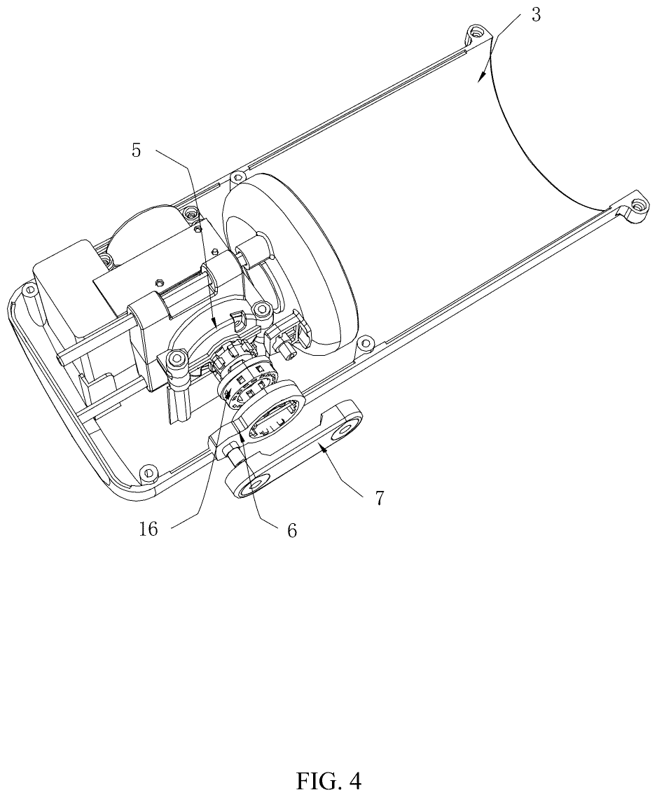

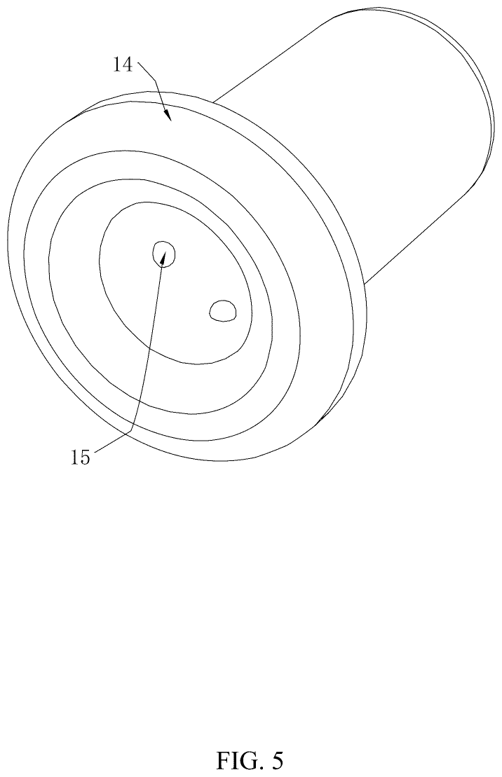

Referring to FIGS. 1 to 5, the present disclosure provides an embodiment of a silicone sperm extractor, including a silicone torso 1, the silicone torso 1 is divided into lower limbs and a waist. A position between the lower limbs is filled with an automatic mechanism, and the automatic mechanism includes an upper shell 2 and a lower shell 3. The upper shell 2 and the lower shell 3 are detachably connected by a plurality of bolts; outer parts of the upper shell 2 and the lower shell 3 abut against an inner wall of the lower limbs of the silicone torso 1. The inner wall of the lower limbs of the silicone torso 1 fully wraps around the upper shell 2 and the lower shell 3. The silicone and the automatic mechanism are molded together by casting, without gaps and having waterproof.

An interior of an inner wall of the lower shell 3 is fixedly connected to a power supply 4, one side of the inner wall of the lower shell 3 close to the power supply 4 is fixedly connected to a motor 5. The power supply 4 and the motor 5 are connected by wires. An output end of the motor 5 is fixedly connected to a cam 6, an inner side of the cam 6 is connected to a noise reduction gear 16 for shock absorption and noise reduction. An outer side of the cam 6 is rotatably connected to a connection rod 7; an inner side of the connection rod 7 is rotatably connected to two transmission wheels 8, one of which is fixedly connected to one side of the cam 6, and an inner side of the other transmission wheel 8 is rotatably connected to a cavity container 9. An outer part of the cavity container 9 is slidably connected to an inner wall of the lower shell 3, a bottom of the cavity container 9 is fixedly connected to two transmission rods 10. An inner wall of the upper shell 2 is fixedly connected with a plurality of through holes 11. An outer side of the transmission rods 10 is slidably connected to an interior of through holes 11. An interior of the cavity container 9 is filled with a silicone simulation organ 14, an inner wall of the silicone simulation organ 14 is provided with a plurality of protrusions 15. The waist of the silicone torso 1 is fixedly connected to a controller 12, an output end of the controller 12 is fixedly connected to a signal line 13. The other end of the signal line 13 is fixedly connected to the motor 5 inside the automatic mechanism, and the signal line 13 is filled inside the silicone torso 1.

Compared with some devices in the existing technology, by simulating human skin to create a strong tactile sensation for a user, and the silicone simulation organ 14 is filled inside. A motion mechanism is provided on an outer side of the silicone simulation organ 14, and the cavity container 9 is driven to reciprocate by a plurality of connection rods 7 outside the motor 5. A plurality of protrusions 15 are provided on an inner wall of the silicone simulation container, so that the user can have a comfortable experience and smoothly and safely extract semen during operation.

Working principle: firstly, the controller 12 is activated by the user, which sends working instructions to the motor 5 through the signal line 13. Upon receiving the instructions, the motor 5 begins to operate, the cam 6 connected to its output end is driven to rotate. With a rotation of the cam 6, the connection rod 7 is moved accordingly, thereby driving two transmission wheels 8 to rotate. One transmission wheel 8 is directly connected to the cam 6, and the other transmission wheel 8 is connected to the cavity container 9 through an internal rotation.

The cavity container 9 moves back and forth along the inner wall of the lower shell 3 under a drive of the transmission wheels 8. The bottom of the cavity container 9 is fixedly connected with two transmission rods 10, which are slidably connected to the plurality of through holes 11 on the inner wall of the upper shell 2. With a movement of the cavity container 9, the transmission rods 10 are slid inside the through holes 11, thereby achieving an effective movement of the cavity container 9.

The interior of the cavity container 9 is filled with the silicone simulation organ 14, and the inner wall of the silicone simulation organ 14 is provided with a plurality of protrusions 15. These protrusions 15 abut against the user's body during a reciprocating motion of the cavity container 9, thereby providing a realistic tactile experience. The design of the overall device aims to simulate the tactile sensation of human skin, allowing the users to have a more comfortable experience during operation.

Ultimately, with the continuous operation of the device, the user can smoothly and safely perform sperm extraction operations, thereby achieving the expected result. The entire process is automated under the control of the motor 5, therefor providing an efficient and user-friendly user experience.

Finally, it should be noted that the above description is only preferred embodiments of the present disclosure and is not intended to limit the present disclosure. Although the present disclosure has been described in detail with reference to the aforementioned embodiments, those skilled in the art may still modify the technical solutions described in the aforementioned embodiments or equivalently replace some of the technical features. Any modifications, equivalent replacements, improvements, etc. made within the spirit and principles of the present disclosure should be included in the protection scope of the present disclosure.

What is claimed is:

1. A silicone sperm extractor, comprising a silicone torso,
wherein the silicone torso is divided into lower limbs and a waist; a position between the lower limbs is filled with an automatic mechanism, and the automatic mechanism comprises an upper shell and a lower shell;
the upper shell and the lower shell are detachably connected by a plurality of bolts; outer parts of the upper shell and the lower shell both abut against an inner wall of the lower limbs of the silicone torso;
an interior of an inner wall of the lower shell is fixedly connected to a power supply;
one side of the inner wall of the lower shell close to the power supply is fixedly connected to a motor;
the motor is provided with a motor protection sleeve, and the protection sleeve is provided with through holes on top and bottom;
the power supply and the motor are connected by wires;
an output end of the motor is fixedly connected to a cam, an inner side of the cam is connected to a noise reduction gear;
an outer side of the cam is rotatably connected to a connection rod,
an interior of the connection rod is connected to two transmission wheels, one of transmission wheels is fixedly connected to one side of the cam, and an interior of the other transmission wheel is rotatably connected a cavity container;
an outer part of the cavity container is slidably connected to on an inner wall of the lower shell;
the cavity container is fixedly connected to two transmission rods,
an outer side of the transmission rods is slidably connected at an interior of the through holes;
an interior of the cavity container is filled with a silicone simulation organ, and an inner wall of the silicone simulation organ is provided with a plurality of protrusions.

2. The silicone sperm extractor according to claim 1, wherein a controller is fixedly connected to one side of the waist of the silicone torso;
a signal line is fixedly connected to an output end of the controller;
the other end of the signal line is fixedly connected to the motor inside the automatic mechanism, and the signal line is filled inside the silicone torso.
